(19) United States
(12) Patent Application Publication
(10) Pub. No.: US 2008/0248993 A1
(43) Pub. Date: Oct. 9, 2008

(54) Modified Beta Thymosin Peptides

(75) Inventors: Ewald Hannappel, Uttenreuth (DE); Thomas Huff, Erlangen (DE); Allan L. Goldstein, Washington, DC (US); David Crockford, Newburyport, MA (US)

Correspondence Address: ROTHWELL, FIGG, ERNST & MANBECK, P.C., 1425 K STREET, N.W., SUITE 800, WASHINGTON, DC, 20005, US

(73) Assignees: REGENERX BIOPHARMACEUTICALS, INC., Bethesda, MD (US); UNIVERSITAET ERLANGEN-NUERNBERG, Erlangen (DE)

(21) Appl. No.: 11/722,979

(22) PCT Filed: Jan. 17, 2006
(86) PCT No.: PCT/US2006/001141

§ 371 Date: Jan. 30, 2008

Related U.S. Application Data
(60) Provisional application No. 60/643,684, filed on Jan. 14, 2005.
(60) Provisional application No. 60/643,686, filed on Jan. 14, 2005.
(60) Provisional application No. 60/643,687, filed on Jan. 14, 2005.
(60) Provisional application No. 60/643,688, filed on Jan. 14, 2005.

(51) Int. Cl. A61K 38/02

(52) U.S. Cl. 514/2

(57) ABSTRACT

A composition including an oxidized or superoxidized methionine-containing beta thymosin peptide, isoform thereof, fragment thereof, isolated R-enantiomer thereof or isolated S-enantiomer thereof, other than racemic thymosin beta 4 sulfoxide, or a modified beta thymosin peptide, isoform or fragment thereof with an amino acid substituent substituted for at least one methionine of an amino acid sequence of a normally methionine-containing beta thymosin peptide, isoform or fragment thereof, and method for forming same.

### CROSS-REFERENCE TO RELATED APPLICATION

This application claims benefit of U.S. Provisional Application Ser. No. 60/643,684, U.S. Provisional Application No. 60/643,686, U.S. Provisional Application Ser. No. 60/643,687 and U.S. Provisional Application Ser. No. 60/643,688, all filed Jan. 14, 2005.

### BACKGROUND

1. Technical Field

The present disclosure relates to the field of beta thymosin peptides, isoforms and fragments thereof.

2. Description of the Background Art

Thymosin β4 was initially identified as a protein that is up-regulated during endothelial cell migration and differentiation in vitro. Thymosin β4 was originally isolated from the thymus and is a 43 amino acid, 4.9 kDa ubiquitous polypeptide identified in a variety of tissues. Several roles have been ascribed to this protein including a role in a endothelial cell differentiation and migration, T cell differentiation, actin sequestration and vascularization.

The amino acid sequence of Tβ4 is disclosed in U.S. Pat. No. 4,297,276, herein incorporated by reference. Tβ4 was highly conserved during evolution. In fact, total homology exists between murine, rat and human Tβ4.

Tβ4 has been found to be present in numerous tissue types in mammals and has also been implicated in a wide variety of cellular and physiological processes including inducing terminal deoxynucleotidyl transferase activity of bone marrow cells, stimulating secretion of hypothalamic luteinizing hormone releasing hormone and luteinizing hormone, inhibiting migration and enhancing antigen presentation of macrophages, and inducing phenotypic changes in T-cell lines in vitro.

Thymosin beta 4 sulfoxide is disclosed in PCT International Publication No. WO 99/49883.

There remains a need in the art for improved beta thymosin peptides.

### SUMMARY

In accordance with one embodiment, a composition comprises an oxidized or superoxidized modified normally methionine-containing beta thymosin peptide, isoform thereof, fragment thereof, isolated R-enantiomer thereof or isolated S-enantiomer thereof, other than racemic thymosin beta 4 sulfoxide, or the composition comprises a modified beta thymosin peptide, isoform or fragment thereof, having a non-methionine amino acid substituent substituted for at least one methionine of an amino acid sequence of a normally methionine-containing beta thymosin peptide, isoform or fragment thereof. Also disclosed are methods for forming a composition in accordance with the present invention.

### DETAILED DESCRIPTION

Many beta thymosin peptides include in their amino acid sequences the amino acid methionine, which is subject to oxidation in vivo and in vitro. Such beta thymosin peptides sometimes are referred to herein as “normally methionine-containing beta thymosin peptides”. In many of the known beta thymosins, methionine is present at position 6.

The oxidation of amino acid, methionine (C₅H₁₁NO₂S), to methionine sulfoxide (C₅H₁₁NO₃S), in normally methionine-containing beta thymosins, results in compositions which are beta thymosin sulfoxides. The oxidation can be accomplished utilizing any suitable method. For example, oxidation of methionine-containing beta thymosins to sulfoxides can be accomplished by exposing the methionine-containing beta thymosins to hydrogen peroxide. Oxidation of thymosin beta 4 with 50 vol. hydrogen peroxide is disclosed in WO 99/4988, incorporated herein by reference. Thus, Thymosin beta 4 can be oxidized by dilute hydrogen peroxide to form thymosin beta 4 sulfoxide as described in WO 99/49883.

The oxidation of amino acid, methionine (C₅H₁₁NO₂S), to methionine sulfoxide (C₅H₁₁NO₃S), or otherwise, also may represent a major degradation pathway of methionine-containing beta thymosins such as Tβ4, both in vivo and in vitro.

Many beta thymosins and isoforms have been identified and have about 70%, or about 75%, or about 80% or more homology to the known amino acid sequence of Tβ4. Such beta thymosins and isoforms include, for example, Tβ4^{ala}, Tβ9, Tβ10, Tβ11, Tβ12, Tβ13, Tβ14 and Tβ15.

Exemplary beta thymosins containing methionine at position 6 include Tβ4, Tβ4, Tβ4^{xen}, Tβ9^{met}, Tβ10 and Tβ13.

The invention is applicable to known beta thymosins, isoforms, and fragments thereof, such as those listed above, as well as normally methionine-containing beta thymosins and Tβ4 isoforms, as well as fragments thereof, not yet identified.

Additionally, the disclosure is applicable to beta thymosins, isoforms and fragments thereof, known or not yet identified, which normally have one or more methionines at a location in the peptide other than at position 6.

In certain embodiments, an amino acid substituted for methionine is neutral, non-polar, hydrophobic and/or non-oxidizing. Such compositions have advantages in greater stability than methionine-containing beta thymosins, while possessing activity substantially the same as, or different from the corresponding beta thymosin.

In certain embodiments, an amino acid being substituted for methionine inhibits oxidation of the beta thymosin, and most preferably, the biological activity of the substituted beta thymosin is substantially the same as that of the corresponding methionine-containing beta thymosin.

Replacement of methionine in a methionine-containing beta thymosin peptide appears to result in a change in the stability profile of the peptide. Additionally, such replacement appears to result in unpredictably new properties of the peptide, as well as unpredictably unchanged properties.

As non-limiting examples, an amino acid to be substituted for methionine in the methionine-containing beta thymosin is valine, isoleucine, alanine, phenylalanine, proline or leucine.

In one embodiment, leucine is substituted for methionine in Tβ4.

According to one embodiment, when leucine is substituted for methionine, the beta thymosin peptide is other than Tβ4.

In accordance with one aspect, the amino acid to be substitute for methionine in the methionine-containing beta thymosin is other than leucine. In some embodiments of this aspect, the amino acid to be substituted for methionine in the methionine-containing beta thymosin is valine, isoleucine, alanine, phenylalanine or proline.

In accordance with one embodiment, the preferred amino acid to be substituted for methionine is valine or isoleucine.

In accordance with another embodiment, the preferred amino acid to be substituted for methionine is alanine.

In accordance with a still further embodiment, the preferred amino acid to be substituted for methionine is valine.

Amino acid-substituted modified beta thymosin peptides, isoforms and fragments thereof in accordance with the present invention can be provided by any suitable method, such as by solid phase peptide synthesis, one example of which is disclosed in U.S. Pat. No. 5,512,656.

The disclosure also is applicable to methods for forming amino acid-substituted modified beta thymosin peptides, wherein the amino acid sequence of a methionine-containing beta thymosin peptide, isoform or fragment thereof is modified by substituting a non-methionine amino acid for at least one methionine in the beta thymosin peptide, isoform or fragment thereof. The method involves substituting a non-methionine amino acid for at least one methionine in a methionine-containing beta thymosin peptide sequence, isoform or fragment thereof so as to form a modified beta thymosin peptide, isoform or fragment thereof.

As noted above, Thymosin beta 4 may have a leucine substituent substituted for methionine at position 6 thereof, so as to comprise Tβ4^{leu}.

The amino acid leucine being substituted for methionine, inhibits oxidation of the Tβ4. Tβ4^{leu} has advantages in greater stability than Tβ4, while surprisingly possessing substantial actin-binding activity.

Replacement of methionine with leucine in Tβ4 may also result in unexpectedly new properties of the peptide.

Methods for forming Tβ4^{leu} are disclosed, wherein the amino acid sequence of Tβ4 is modified by substituting a leucine amino acid for the methionine in Tβ4 at position 6. This method involves substituting a leucine for methionine in the Tβ4 sequence at position 6, so as to form Tβ4^{leu}.

Peptides in accordance with the invention may possess substantial actin-binding activity.

The compositions may be utilized, among other things, as anti-inflammatory agents.

In accordance with one embodiment, an oxidized methionine-containing beta thymosin peptide, isoform or fragment thereof is provided, other than thymosin beta 4 sulfoxide.

Also is applicable are methods for forming modified beta thymosin sulfoxides comprising contacting a normally methionine-containing beta thymosin peptide, isoform or fragment thereof with an oxidizing agent such as dilute hydrogen peroxide, to form a beta thymosin sulfoxide peptide.

Another embodiment is a method of forming a beta thymosin sulfoxide peptide comprising contacting a normally methionine-containing beta thymosin peptide, isoform or fragment thereof, other than thymosin beta 4, with an oxidizing agent such as hydrogen peroxide to form a corresponding beta thymosin sulfoxide peptide.

The compositions included herein have advantages in greater stability than non-oxidised methionine-containing beta thymosins, while possessing activity substantially the same as, or different from the corresponding beta thymosin.

Oxidation of a methionine-containing beta thymosin peptide to a sulfoxide results in a change in the stability profile of the peptide. Additionally, such replacement may result in certain new properties of the peptide, as well as certain unchanged properties.

Superoxidation of methionine in a normally methionine-containing beta thymosin peptide results in a change in the stability profile of the peptide. Additionally, such replacement may result in certain new properties of the peptide, as well as certain unchanged properties.

According to one embodiment, the beta thymosin peptide to be superoxidised is other than Tβ4.

Methionine-containing beta thymosin peptides, including beta thymosin sulfoxides, can be superoxidized by performic or peracetic acid to form a corresponding beta thymosin sulfone peptide, in which the sulfur atom of the affected methionine is fully oxidized (superoxidised) with two oxygens.

In accordance with one embodiment, a composition is provided comprising a modified methionine-containing beta thymosin sulfone other than Tβ4 sulfone.

Included are methods for forming a beta thymosin sulfone comprising contacting a methionine-containing beta thymosin peptide, sulfoxide, isoform or fragment thereof with an acid such as performic and/or peracetic acid to form a corresponding beta thymosin sulfone peptide.

In accordance with another embodiment, included is a method of forming a beta thymosin sulfone peptide comprising contacting a methionine-containing beta thymosin peptide, sulfoxide, isoform or fragment thereof, other than thymosin beta 4 or thymosin beta 4 sulfoxide, with an acid such as performic and/or peracetic acid, to form a corresponding beta thymosin sulfone peptide.

In another embodiment, thymosin beta 4 and/or thymosin beta 4 sulfoxide is oxidized by performic or peracetic acid to form thymosin beta 4 sulfone, in which the sulfur atom of methionine at position 6 of thymosin beta 4 is fully oxidized (superoxidized) with two oxygens.

Thymosin beta 4 sulfone may be utilized, among other things, as an anti-inflammatory agent.

Because the thioether in the methionyl residue of Tβ4 is prochiral, oxidation (or superoxidation) with a non-chiral agent generates two forms of a sulfoxide (or sulfone) namely S- and an R-forms (“enantiomers”). Since every biomolecule being a potential partner of the oxidized thymosin beta 4 is chiral, the complexes formed with the S- or the R-form of the sulfoxide (or sulfone) are different (diastereomers). The forms of thymosin beta 4 sulfoxide (or sulfone) may show different pharmacodynamic and pharmacokinetic behavior as well as biological activities. Thus, the S- and R-forms of thymosin beta 4 sulfoxide (or sulfone) may have different biological effects.

An amino acid analysis system may be used to discriminate between L-methionine (S)- and (R)-sulfoxides (or sulfones) after hydrolysis of oxidized thymosin beta 4 sulfoxide (or sulfone). For example, two procedures may be used to isolate the two forms of thymosin beta 4 sulfoxide (or sulfone) after oxidation or superoxidation. Although the different forms of methionine sulfoxides (or sulfones) behave as mirror and mirror image, the generated thymosin beta 4 sulfoxides (or sulfones) constitute not a pair of image and mirror image because the other amino acid residues of the peptide generate an asymmetric environment. Thus, the separate forms are separable by HPLC techniques. An alternative procedure to separate the forms may be the use of specific methionine sulfoxide (or sulfone) reductases which reduce one form of the thymosin beta 4 sulfoxide (or sulfone) but not the other. The separation of the non-reducible form of thymosin beta 4 sulfoxide (or sulfone) from the enzymatic reduced form may be done by HPLC.

Disclosed herein is a method of forming a composition containing separated R-thymosin beta 4 sulfoxide (or sulfone), by separating R-thymosin beta 4 sulfoxide (or sulfone) from a mixture containing R-thymosin beta 4 sulfoxide (or sulfone) and S-thymosin beta 4 sulfoxide (or sulfone).

Disclosed herein is a method for forming a composition containing separated S-thymosin beta 4 sulfoxide (or sulfone), by separating S-thymosin beta 4 sulfoxide (or sulfone) from a mixture containing S-thymosin beta 4 sulfoxide (or sulfone) and R-thymosin beta 4 sulfoxide (or sulfone).

As noted above, many beta thymosins and isoforms thereof have been identified and have about 70%, or about 75%, or about 80% or more homology to the known amino acid sequence of Tβ4. Such beta thymosins and isoforms include, for example, Tβ4^{ala}, Tβ9, Tβ10, Tβ11, Tβ12, Tβ13, Tβ14 and Tβ15.

As noted above, exemplary beta thymosins containing methionine at position 6 include Tβ4, Tβ4^{ala}, Tβ4^{xen}, Tβ9^{met}, Tβ10 and Tβ13.

As noted above, included herein are known beta thymosins and isoforms, such as those listed above, as well as methionine-containing beta thymosins, isoforms, and fragments thereof, not yet identified.

As noted above, additionally included herein are beta thymosins, isoforms and fragments thereof, known or not yet identified, having one or more methionines at a location in the peptide other than at position 6.

As with Tβ4, because the thioether in the methionyl residue(s) of methionine-containing beta thymosins is prochiral, oxidation (or superoxidation) with a non-chiral agent generates at least two forms of sulfoxide (or sulfone), including an S- and an R-form (“enantiomers”). Since every biomolecule being a potential partner of the oxidized methionine-containing beta thymosins is chiral, the complexes formed with the S- or the R-form of the sulfoxide (or sulfone) are different (diasteromers). The different forms of beta thymosin sulfoxide (or sulfone) show different pharmacodynamic and pharmacokinetic behavior as well as biological activity. Thus, the S- and R-forms of a beta thymosin sulfoxide (or sulfone) may have different biological effects.

As with Tβ4, an amino acid analysis system may be used to discriminate between an L-methionine (S)- and (R)-sulfoxide (or sulfone) after hydrolysis of an oxidized beta thymosin sulfoxide (or sulfone). For example, at least two procedures may be used to isolate the different forms of a beta thymosin sulfoxide (or sulfone) after oxidation (or superoxidation). Although the different forms of methionine sulfoxide (or sulfones) behave as mirror, and mirror image, the generated beta thymosin sulfoxides (or sulfones) constitute not a pair of image, and mirror image, because the other amino acid residues of the peptide(s) generate an asymmetric environment. Thus, the different forms are separable by known HPLC techniques. An alternative procedure to separate the different forms may be the use of specific methionine sulfoxide (or sulfone) reductases which reduce one form of the beta thymosin sulfoxide (or sulfone) but not another. The separation of the non-reducible form of a beta thymosin sulfoxide (or sulfone) from the enzymatic reduced form may be done by HPLC.

Disclosed herein is a method of forming a composition containing a separated R-beta thymosin sulfoxide (or sulfone), by separating an R-beta thymosin sulfoxide (or sulfone) from a mixture containing an R-beta thymosin sulfoxide (or sulfone) and an S-beta thymosin sulfoxide (or sulfone).

Further disclosed is a method for forming a composition containing a separated S-beta thymosin sulfoxide (or sulfone), by separating an S-beta thymosin sulfoxide (or sulfone) from a mixture containing an S-beta thymosin sulfoxide (or sulfone) and an R-beta thymosin beta 4 sulfoxide (or sulfone).

In one embodiment, the disclosure provides a method of treatment for treating, preventing, inhibiting or reducing disease, damage, injury and/or wounding of a subject, or of tissue of a subject, by administering an effective amount of a composition which contains a peptide as described herein. The administering may be directly or systemically. Examples of direct administration include, for example, contacting tissue, by direct application or inhalation, with a carrier comprising a solution, lotion, salve, gel, cream, paste, spray, suspension, dispersion, hydrogel, ointment, or oil including a peptide as described herein. Systemic administration includes, for example, intravenous, intraperitoneal, intramuscular injections of a composition containing a peptide as described herein, in a pharmaceutically acceptable carrier such as water for injection. The subject preferably is mammalian, most preferably human.

Compositions, as described herein, may be administered in any suitable effective amount. For example, a composition as described herein may be administered in dosages within the range of about 0.0001-1,000,000 micrograms, more preferably in amounts within the range of about 0.1-5,000 micrograms, most preferably within the range of about 1-30 micrograms.

A composition as described herein can be administered daily, every other day, every other week, every other month, etc., with a single application or multiple applications per day of administration, such as applications 2, 3, 4 or more times per day of administration.

The disclosure also includes a pharmaceutical or cosmetic composition comprising a therapeutically effective amount of a composition as described herein in a pharmaceutically or cosmetically acceptable carrier. Such carriers include any suitable carrier, including those listed herein.

The approaches described herein involve various routes of administration or delivery of a composition as described herein, including any conventional administration techniques (for example, but not limited to, direct administration, local injection, inhalation, or systemic administration), to a subject. The methods and compositions using or containing a composition as described herein may be formulated into pharmaceutical or cosmetic compositions by admixture with pharmaceutically acceptable or cosmetically non-toxic excipients, additives or carriers.

### EXAMPLE 1

Tβ4^{val} is produced with valine substituted for methionine at position 6, by conventional solid phase peptide synthesis, e.g., according to the method disclosed in U.S. Pat. No. 5,512,656, resulting in a peptide having unexpected and unpredictable properties.

### EXAMPLE 2

Tβ4^{iso} is produced with isoleucine substituted for methionine at position 6, by conventional solid phase peptide synthesis, resulting in a peptide having unexpected and unpredictable properties.

### EXAMPLE 3

Tβ4 M6A is produced with alanine substituted for methionine at position 6, by conventional solid phase peptide synthesis, e.g., according to the method disclosed in U.S. Pat. No. 5,512,656, resulting in a peptide having unexpected and unpredictable properties.

### EXAMPLE 4

Tβ4^{phe} is produced with phenyalonine substituted for methionine at position 6, e.g., according to the method disclosed in U.S. Pat. No. 5,512,656, by conventional solid phase peptide synthesis, resulting in a peptide having unexpected and unpredictable properties.

### EXAMPLE 5

Tβ4^{pro} is produced with proline substituted for methionine at position 6, e.g., according to the method disclosed in U.S. Pat. No. 5,512,656, by conventional solid phase peptide synthesis, resulting in a peptide having unexpected and unpredictable properties.

### EXAMPLE 6

Tβ4^{leu} is produced with leucine substituted for methionine at position 6, e.g., according to the method disclosed in U.S. Pat. No. 5,512,656, by conventional solid phase peptide synthesis, resulting in a peptide having unexpected and unpredictable properties.

### EXAMPLE 7

Tβ4^{leu} was produced by solid phase peptide synthesis as described herein.

Tests were conducted to determine the G-actin binding affinity of Tβ4^{leu} as compared to native Tβ4. The experiments were repeated twice and in triplicate, with the results shown below (averages from 3 experiments):

First determination: Tβ4 Kd=1.28 microM; Tβ4^{leu} Kd=1.36 microM

Second determination: Tβ4 Kd=0.54 microM; Tβ4^{leu} Kd=0.99 microM

The lower values in the case of Tβ4 are caused by its sulfoxide. Tβ4 plus Tβ4-sulfoxide was determined by amino acid analysis. About 10% in the preparation was sulfoxide which binds only weakly to G-actin. Thus the real concentration of Tβ4 in the tests are lower and the decrease of free Tβ4 is higher when compared to non-oxidized Tβ4. The decrease of free beta-thymosin was measured. G-actin bound Tβ4 and free Tβ4 were separated by ultrafiltration and the concentration of Tβ4 in the ultrafiltrate was measured by HPLC. The procedure utilized is described in Huff et al., FEPS Letters, 414:39-44 (1997).

The above data indicates surprisingly that amino acid substitution for methionine in a beta thymosin peptide does not substantially adversely affect the G-actin binding capabilities of the beta thymosin peptide.

### EXAMPLE 8

Tβ4 sulfone was produced by complete oxidation of the Met residue of Tβ4 by treatment of Tβ4 with concentrated (30%) H₂O₂.

The chemical nature of Tβ4 sulfone has been established. The techniques used were HPLC, MALDI-TOF MS and amino acid analysis. MALDI-TOF analysis showed an increase of the molecular mass of the peptide of 32 Da which corresponds to the incorporation of O₂ into the peptide.

Tβ4-sulfone has been characterized in terms of binding of G-actin. It forms a complex with G-actin and the Kd value of the complex is about 10 uM. Therefore the complex is less stable compared to the complex with Tβ4 (1 uM) but surprisingly more stable than a complex with Tβ4-sulfoxide (20 uM).

### EXAMPLE 9

Beta thymosins including Tβ4^{ala}, Tβ4^{xen}, Tβ9^{met}, Tβ10 and Tβ13 are converted to sulfoxides by contacting with dilute hydrogen peroxide as described herein to form corresponding beta thymosin sulfoxides.

### EXAMPLE 10

Beta thymosins including Tβ4^{ala}, Tβ4^{xen}, Tβ9^{met}, Tβ10 and Tβ13 are converted to beta thymosin sulfone peptides by contacting them with performic and/or peracetic acid as described herein, so as to form the corresponding beta thymosin sulfone peptides.

### EXAMPLE 11

Tβ4 sulfoxide, which is a one-to-one mixture of the S- and the R-form, has been reduced by specific methionine sulfoxide reductases (MSR-A and MSR-B). By this procedure either the S-form or R-form were reduced while the other form stayed as sulfoxide. The procedure provided two samples: one sample containing a mixture of Tβ4 and R-Tβ4 sulfoxide and the other sample containing a mixture of Tβ4 and S-Tβ4 sulfoxide. The two samples were separated by HPLC and pure S-Tβ4 sulfoxide and R-Tβ4 sulfoxide were isolated. The dissociation constants of their complexes with G-actin were determined. Surprisingly, the dissociation constants of their complexes were identical. The stabilities of the complexes of G-actin with either (R/S)-Tβ4 sulfoxide or R-Tβ4 sulfoxide or S-Tβ4 sulfoxide are identical (Kd˜20 uM). The Kd of the actin-Tβ4 sulfoxide is about 1 uM. It is possible that R-Tβ4 sulfoxide is converted to S-Tβ4 sulfoxide (and S-Tβ4 sulfoxide to R-Tβ4 sulfoxide) by binding to G-actin. This racemisation would abolish differences in the Kd values.

### EXAMPLE 12

Tβ4-sulfone produced according to Example 8, as well as beta thymosin sulfoxides produced according to Example 9 and beta thymosin sulfones produced according to Example 10 are separated to form respective isolated S-enantiomers and R-enantiomers thereof.

1. A composition comprising an oxidized or superoxidized modified normally methionine-containing beta thymosin peptide, isoform thereof, fragment thereof, isolated R-enantiomer thereof or isolated S-enantiomer thereof, other than racemic thymosin beta 4 sulfoxide, or comprising a modified beta thymosin peptide, isoform or fragment thereof having a non-methionine amino acid substituent substituted for at least one methionine of an amino acid sequence of a normally methionine-containing beta thymosin peptide, isoform or fragment thereof.

2. The composition of claim 1 wherein said normally methionine-containing beta thymosin peptide is Tβ4, Tβ4^{ala}, Tβ4^{xen}, Tβ9^{met}, Tβ10 or Tβ13.

3. The composition of claim 1 wherein said amino acid substituent is leucine, valine, isoleucine, alanine, phenylalanine or proline.

4. The composition of claim 1 wherein said methionine-containing beta thymosin peptide is Tβ4.

5. The composition of claim 1 wherein said amino acid substituent is leucine, valine, isoleucine, alanine, phenylalanine or proline.

6. The composition of claim 1 wherein said amino acid substituent is at least one of neutral, non-polar, hydrophobic or non-oxidizing.

7. The composition of claim 1 wherein said amino acid substituent inhibits oxidation of said peptide.

8. The composition of claim 1 comprising a modified beta thymosin peptide, isoform or fragment thereof having a non-methionine amino acid substituent other than leucine substituted for at least one methionine of an amino acid sequence of a methionine-containing beta thymosin peptide other than Tβ4, or an isoform or fragment of a normally methionine-containing beta thymosin peptide.

9. The composition of claim 1 comprising peptide Tβ4^{leu}, which comprises a modified Tβ4 amino acid sequence having leucine substituted for methionine at position 6 thereof.

10. The composition of claim 1 wherein the modified methionine-containing beta thymosin peptide, isoform or fragment thereof is a sulfoxide.

11. The composition of claim 1 wherein the modified methionine-containing beta thymosin peptide, isoform or fragment thereof is a sulfone.

12. The composition of claim 1 comprising thymosin beta 4 sulfone.

13. The composition of claim 1 comprising Tβ4^{ala} sulfone, Tβ4^{xen} sulfone, Tβ4^{met} sulfone, Tβ10 sulfone or Tβ13 sulfone.

14. The composition of claim 1 comprising an isolated R-enantiomer.

15. The composition of claim 1 comprising an isolated S-enantiomer.

16. The composition of claim 1 comprising an isolated R-enantiomer of thymosin beta 4 sulfoxide.

17. The composition of claim 1 comprising an isolated S-enantiomer of thymosin beta 4 sulfoxide.

18. A method for forming a beta thymosin sulfone peptide according to claim 1, comprising contacting a normally methionine-containing beta thymosin peptide with an acid, so as to form a beta thymosin sulfone peptide.

19. The method of claim 18 wherein said acid comprises at least one of performic acid or peracetic acid.

20. A method of forming a composition according to claim 1, comprising an isolated R-beta thymosin sulfoxide or sulfone, the method comprising separating an R-beta thymosin sulfoxide or sulfone from a mixture of corresponding R-beta thymosin sulfoxide or sulfone and S-beta thymosin sulfoxide or sulfone.

21. A method of forming a composition according to claim 1, comprising an isolated S-beta thymosin sulfoxide or sulfone, the method comprising separating an S-beta thymosin sulfoxide or sulfone from a mixture containing corresponding S-beta thymosin sulfoxide or sulfone and R-beta thymosin sulfoxide or sulfone.

22. A method for forming a modified beta thymosin peptide, isoform or fragment thereof as defined in claim 1, comprising substituting a non-methionine amino acid substituent for at least one methionine of amino acid sequence of a normally methionine-containing beta thymosin peptide, isoform or fragment thereof, so as to form a modified beta thymosin peptide, isoform or fragment thereof.

23. The method of claim 22 wherein said amino acid substituent is leucine, valine, isoleucine, alanine, phenylalanine or proline.

24. The method of claim 22 for forming peptide Tβ4^{leu}, comprising substituting amino acid leucine for methionine at position 6 of Tβ4 so as to form peptide Tβ4^{leu}.
